(19) United States
(12) Patent Application Publication
(10) Pub. No.: US 2026/0083691 A1
(43) Pub. Date: Mar. 26, 2026

(54) TREATING CANCERS WITH COMBINATIONS OF ANTI-CD20 ANTIBODY AND ACYLFULVENES

(71) Applicant: Lantern Pharma Inc., Dallas, TX (US)

(72) Inventors: Jianli Zhou, Dallas, TX (US); Kishor Bhatia, Atlanta, GA (US)

(73) Assignee: Lantern Pharma Inc., Dallas, TX (US)

(21) Appl. No.: 19/405,201

(22) Filed: Dec. 1, 2025

Related U.S. Application Data
(63) Continuation of application No. PCT/US2024/031675, filed on May 30, 2024.
(60) Provisional application No. 63/504,949, filed on May 30, 2023.

(51) Int. Cl. A61K 31/17; A61K 31/337; A61K 33/243; A61K 39/00; A61P 35/00; C07K 16/28

(52) U.S. Cl. CPC A61K 31/17; A61K 31/337; A61K 33/243; A61P 35/00; C07K 16/2887; A61K 2039/505; A61K 2039/545

(57) ABSTRACT

A method of treating cancer includes administering a combination of active agents comprising a therapeutically effective amount of an acylfulvene, or a pharmaceutically acceptable salt thereof, and a therapeutically effective amount of an anti-CD20 antibody. The anti-CD20 antibody can be selected from rituximab, obinutuzumab, ofatumumab, and tositumomab. The cancer may be a B-cell cancer.

### CROSS-REFERENCE TO RELATED APPLICATIONS

This is application a continuation of International Patent Application No. PCT/US2024/031675, filed on May 30, 2024, claims the benefit of U.S. Provisional Patent Application No. 63/504,946, filed May 30, 2023, each of which is incorporated by reference herein in its entirety.

### TECHNICAL FIELD

This application relates to cancer treatments and more specifically this application relates to cancer treatments (e.g., lymphomas) using a combination therapy including rituximab and an acylfulvene.

### BACKGROUND

Non-Hodgkin's lymphomas (NHL) are a group of cancers that originate in the lymphatic system, which is part of the body's immune system. Unlike Hodgkin's lymphoma, which is characterized by the presence of Reed-Sternberg cells, NHL comprises a diverse set of lymphoid malignancies with different subtypes and characteristics.

NHL is classified into various subtypes based on the specific type of lymphocyte involved (B cells, T cells, or natural killer cells), the cell's maturity stage, and other factors such as genetic mutations and growth patterns. Common subtypes include diffuse large B-cell lymphoma (DLBCL), which is the most prevalent and aggressive form; follicular lymphoma (FL), which typically grows more slowly; mantle cell lymphoma (MCL), known for its poor prognosis and aggressive nature; and Burkitt lymphoma, a highly aggressive cancer associated with the Epstein-Barr virus. Each subtype has distinct clinical presentations, prognoses, and treatment approaches.

Anti-CD20 antibodies have revolutionized the treatment of various diseases by specifically targeting B cells and modulating the immune response. These monoclonal antibodies, such as rituximab, work by binding to the CD20 protein on the surface of B cells, leading to their destruction through mechanisms like antibody-dependent cellular cytotoxicity (ADCC), complement-dependent cytotoxicity (CDC), and direct apoptosis. This targeted approach has significantly improved outcomes for patients with B-cell malignancies, including many subtypes of NHL. However, anti-CD20 monoclonal antibody alone typically cannot cure B-cell malignancies, especially the more aggressive forms.

Accordingly, there is always a need for improved treatments for NHL and other blood cancers. It is to this need, among others, that this application is directed.

### SUMMARY

One aspect includes a method of treating cancer that involves administering to a subject in need of treatment a combination of active agents, specifically a therapeutically effective amount of an acylfulvene or a pharmaceutically acceptable salt thereof, and a therapeutically effective amount of an anti-CD20 antibody. The acylfulvene can be HydroxyUreaMethyl Acylfulvene. The acylfulvene have the following structure:

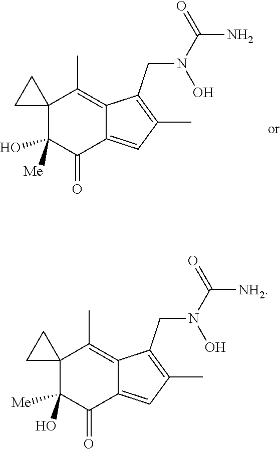 [SMILES: CC1=C(CN(O)C(N)=O)C2=C(C)C3(CC3)[C@@](C)(O)C(=O)C2=C1 ; CC1=C(CN(O)C(N)=O)C2=C(C)C3(CC3)[C@](C)(O)C(=O)C2=C1]

Another aspect includes a method in which the anti-CD20 antibody is selected from the group consisting of rituximab, obinutuzumab, ofatumumab, and tositumomab. The anti-CD20 antibody can be rituximab or an antigen-binding fragment of the antibody.

Another aspect includes a method in which the blood cancer being treated is a B-cell nonHodgkin's lymphoma, including diffuse large B-cell lymphoma (DLBCL), follicular lymphoma (FL), mantle cell lymphoma (MCL), or Burkitt lymphoma.

Another aspect includes a method where the administration of the acylfulvene and the anti-CD20 antibody is sequential or simultaneous.

Another aspect includes a method in which the acylfulvene and the anti-CD20 antibody are formulated for intravenous administration.

Another aspect includes a method where the combination therapy results in enhanced apoptosis of cancerous B cells compared to the administration of either the illudin analog or the anti-CD20 antibody alone.

Another aspect includes a method in which the presence of one or more cancer cells deficient in HR-dependent DNA Damage repair indicates that the subject will respond to the treatment.

Another aspect includes a method of administering a pharmaceutically acceptable carrier or excipient along with the illudin analog and the anti-CD20 antibody.

Another aspect includes a method in which the combination therapy reduces the proliferation of cancerous B cells and inhibits tumor growth.

Another aspect includes a method of treating B-cell cancer that is relapsed or refractory.

### BRIEF DESCRIPTION OF THE DRAWINGS

FIG. 1 illustrates the synergistic effect of LP-284 or (+)-hydroxyureamethyl acylfulvene in combination with an anti-CD20 monoclonal antibody (Rituximab) on tumor growth in a xenograft model;

FIG. 2 depicts the tumor growth curves resulting from treatment with LP-284 or (+)-hydroxyureamethyl acylfulvene alongside the lymphoma standard-of-care agent Rituximab; and

FIG. 3 demonstrates that LP-284 treatment significantly reduced B-cells (CD19+ or CD20+) by approximately 30% in immune-proficient mice after three doses.

### DETAILED DESCRIPTION

This application provides a combination therapy for treating blood cancers. In embodiments, the therapy includes administering a combination of agents including an acylfulvene or an illudin and an anti-CD20 antibody. In other embodiments, the therapy includes administering a combination of other therapies. In other embodiments, the therapy includes the combination therapy that can be used to treat biochemical occurrence and recurrence of blood cancers in which an acylfulvene (e.g., hydroxyureamethyl acylfulvene) or salt thereof and an anti-CD20 antibody administered in a therapeutically effective amount to the patient. In certain embodiments, the combination can provide a treatment for lymphoma, such as mantle cell lymphoma (MCL) and double-hit lymphoma (DHL). Tn specific embodiments, the combination is used to treat nonHodgkin's lymphomas that are homologous recombination deficiency positive (HRD+).

### Illudin or Acylfulvene

In one embodiment, this application includes the use of an illudin or illudin analog (e.g., acylfulvene). Acylfulvene is a class of cytotoxic semi-synthetic derivatives of illudin, a natural product that can be extracted from the jack o'lantern mushroom (Omphalotus olearius). Acylfulvene, derived from the sesquiterpene illudin S by treatment with acid (reverse Prins reaction), is far less reactive to thiols than illudin S.

In one example, the acylfulvene is (−)-hydroxyureamethyl acylfulvene (termed LP-184 by Lantern Pharma Inc.), which shifts light positively, is shown below:

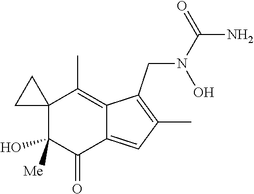 [SMILES: CC1=C(CN(O)C(N)=O)C2=C(C)C3(CC3)[C@@](C)(O)C(=O)C2=C1]

In another example, the acylfulvene is (+)-hydroxyureamethyl acylfulvene (termed LP-284 by Lantern Pharma Inc.), which shifts light negatively, is shown below:

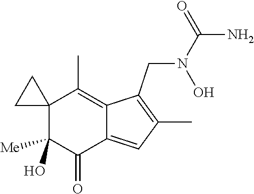 [SMILES: CC1=C(CN(O)C(N)=O)C2=C(C)C3(CC3)[C@](C)(O)C(=O)C2=C1]

(+)-hydroxyureamethyl acylfulvene and (−)-hydroxyureamethyl acylfulvene are enantiomers and are now known publicly.

In another example, the acylfulvene is Irofulven.

### Anti-CD20 Antibody

Anti-CD20 antibodies are a type of monoclonal antibody that specifically target the CD20 protein found on the surface of certain immune cells called B cells. These antibodies are designed to selectively bind to CD20, leading to various therapeutic effects. The term “anti-CD20 antibody” refers to an antibody that binds specifically to the CD20 antigen. Depending on the binding properties and biological activities of anti-CD20 antibodies to the CD20 antigen, there are two main types of anti-CD20 antibodies.

One specific and well-known anti-CD20 antibody is rituximab, which was the first approved monoclonal antibody targeting CD20. Rituximab is used in the treatment of several conditions, including non-Hodgkin's lymphoma (NHL), chronic lymphocytic leukemia (CLL), rheumatoid arthritis (RA), and certain autoimmune disorders. Rituximab works by targeting the CD20 protein, which is found on the surface of B cells. By binding to CD20, rituximab helps to selectively destroy B cells, which can be overactive or cancerous in certain conditions. For non-Hodgkin lymphoma (NHL), rituximab is often used in combination with chemotherapy. For chronic lymphocytic leukemia (CLL), rituximab is sometimes used in combination with other drugs to treat this type of cancer affecting white blood cells.

Rituximab is a genetically engineered chimeric murine/human monoclonal antibody directed against the CD20 antigen, as described in U.S. Pat. No. 5,736,137 issued Apr. 7, 1998 (Anderson et al.).

In specific embodiments, an anti-CD20 antibody may comprise or consist of a rituximab biosimilar, such as blitzima, ritemvia, or tuxella. Rituximab and its biosimilars belong to a class of drugs known as monoclonal antibodies and are primarily used to treat certain types of cancers and autoimmune diseases.

In one embodiment, rituximab can be administered intravenously, and the treatment protocol can vary depending on the condition being treated.

### Homologous Recombination Deficiency (HRD) Positive

Homologous recombination deficiency (HRD) positivity refers to the presence of genetic or genomic alterations in a tumor that indicate a defect or impairment in the homologous recombination DNA repair pathway. HRD positivity is often assessed in the context of cancer, particularly in relation to predicting response to certain treatments.

When tumors have HRD, they are more likely to have difficulties repairing DNA damage, such as double-stranded breaks. This can make them more susceptible to certain therapies that exploit these repair deficiencies.

HRD positivity can be determined through various methods, including genetic testing, genomic profiling, or specific biomarker assays. These tests aim to identify genetic or genomic alterations associated with HRD, such as mutations or loss of function in genes involved in the homologous recombination pathway.

Assessing HRD positivity can have implications for treatment decisions. For example, in ovarian cancer, HRD positivity has been used as a predictive biomarker for the response to a drug. Drugs that block an alternative DNA repair pathway relied upon by HRD-positive cancer cells, leading to their selective targeting and cell death.

In one embodiment, acylfulvene or hydroxyureamethyl acylfulvene or its salt may be administered either prior to, concomitantly with, or subsequent to the administration of an anti-CD20 antibody (e.g., rituximab).

One aspect of this application includes a method of treating cancer in a subject in need thereof. The method involves administering to the subject an effective amount of an anti-CD20 antibody (e.g., rituximab) and an effective amount of an acylfulvene. The anti-CD20 antibody (e.g., rituximab) may be administered prior to or concomitantly with an acylfulvene for optimal synergistic effects.

Another embodiment includes a pharmaceutical composition having a therapeutically effective amount of an illudin or an illudin analog thereof, derivative, or a pharmaceutically acceptable salt thereof; and a therapeutically effective amount of an anti-CD20 antibody (e.g., rituximab) or an analog, derivative, or a pharmaceutically acceptable salt thereof. The illudin analog can be hydroxyureamethyl acylfulvene.

In another embodiment, a kit for the treatment of cancer in a subject includes a therapeutically effective amount of an illudin or an illudin analog thereof, derivative, or a pharmaceutically acceptable salt thereof; and a therapeutically effective amount of anti-CD20 antibody (e.g., rituximab).

In another embodiment, the second therapeutic is one or more chemotherapeutic agents selected from camptothecin derivatives, paclitaxel, docetaxel, epothilone B, 5-FU, gemcitabine, oxaliplatin, cisplatinum, carboplatin, melphalam, dacarbazine, temozolomide, doxorubicin, imatinib, erlotinib, bevacizumab, cetuximab and a Raf kinase inhibitor.

In another embodiment, the second therapeutic is one or more chemotherapeutic agents selected from paclitaxel or cisplatinum.

In one embodiment, acylfulvene or hydroxyureamethyl acylfulvene or its salt may be administered either prior to, concomitantly with, or subsequent to the administration of an anti-CD20 antibody.

One aspect of this application includes a method of treating cancer in a subject in need thereof. The method involves administering to the subject an effective amount of an anti-CD20 antibody and an effective amount of an acylfulvene. An anti-CD20 antibody may be administered prior to or concomitantly with an acylfulvene for optimal synergistic effects. Anti-CD20 antibodies degrade the key Nucleotide Excision Repair (NER) protein XPB/ERCC3 (reference: https://www.ncbi.nlm.nih.gov/pmc/articles/PMC7277409/) and lead to NER deficiency. Subjects with NER deficiency are more sensitive to an illudin-based anti-cancer agent.

Another embodiment includes a pharmaceutical composition having a therapeutically effective amount of an illudin or an illudin analog thereof, derivative, or a pharmaceutically acceptable salt thereof; and a therapeutically effective amount of an anti-CD20 antibody or an analog, derivative, or a pharmaceutically acceptable salt thereof. The illudin analog can be HydroxyUreaMethyl Acylfulvene.

In another embodiment, a kit for the treatment of cancer in a subject includes a therapeutically effective amount of an illudin or an illudin analog thereof, derivative, or a pharmaceutically acceptable salt thereof; and a therapeutically effective amount of an anti-CD20 antibody or an analog, derivative, or a pharmaceutically acceptable salt thereof.

In another embodiment, the second therapeutic is one or more chemotherapeutic agents selected from camptothecin derivatives, paclitaxel, docetaxel, epothilone B, 5-FU, gemcitabine, oxaliplatin, cisplatinum, carboplatin, melphalan, dacarbazine, temozolomide, doxorubicin, imatinib, erlotinib, bevacizumab, cetuximab, and a Raf kinase inhibitor.

In another embodiment, the second therapeutic is one or more chemotherapeutic agents selected from paclitaxel or cisplatinum.

The term “combination therapy” can include or includes the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In another aspect, a composition or combination therapy herein, or a pharmaceutically acceptable salt or solvate thereof, may be administered in combination with radiation therapy. Radiation therapy can also be administered in combination with a composition of the present invention and another chemotherapeutic agent described herein as part of a multiple agent therapy.

Combination therapy can be achieved by administering two or more agents, e.g., an acylfulvene, an anti-CD20 antibody, and one or more other therapeutic agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present within the patient's body at the same time, this need not be so.

The methods of combination therapy may or should result in a synergistic effect, wherein the effect of a combination of compounds or other therapeutic agents is greater than the sum of the effects resulting from administration of any of the compounds or other therapeutic agents as single agents. A synergistic effect may also be an effect that cannot be achieved by administration of any of the compounds or other therapeutic agents as single agents. The synergistic effect may include, but is not limited to, an effect of treating cancer by reducing tumor size, inhibiting tumor growth, or increasing survival of the subject. The synergistic effect may also include reducing cancer cell viability, inducing cancer cell death, and inhibiting or delaying cancer cell growth.

Therapeutically effective doses can vary, as recognized by those skilled in the art, depending on the diseases treated, the severity of the disease, the route of administration, the age and general health condition of the patient, excipient usage, the possibility of co-usage with other therapeutic treatments such as use of other agents and the judgment of the treating physician. For example, guidance for selecting an effective dose can be determined by reference to the prescribing information for hydroxyureamethyl acylfulvene or journal discussion of the same.

The term “effective amount” as used herein refers to the amount of an agent needed to alleviate at least one or more symptoms of the disease or disorder, and relates to a sufficient amount of pharmacological composition to provide the desired effect. The term “therapeutically effective amount” therefore refers to an amount of the agent that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

The dosage ranges for the administration of an agent according to the methods described herein depend upon, for example, the form of the agent, its potency, and the extent to which symptoms, markers, or indicators of a condition described herein are desired to be reduced, for example, the percentage reduction desired for tumor growth. The dosage should not be so large as to cause adverse side effects. Generally, the dosage will vary with the age, condition, and sex of the patient and can be determined by one of skill in the art. The dosage can also be adjusted by the individual physician in the event of any complication.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

The efficacy of an agent described herein in, e.g., the treatment of a condition described herein, or to induce a response as described herein (e.g., solid cancers or blood cancers) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e g., tumor size and/or growth rate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example, treatment of blood cancers in a mouse model. When using an experimental animal model, efficacy of treatment is evidenced when a statistically significant change in a marker is observed, e.g. tumor size and/or growth rate. In some embodiments, the therapeutically effective amount of hydroxyureamethyl-acylfulvene, acyl fulvenes, or Irofulven or a pharmaceutically acceptable salt thereof is selected from the group consisting of 0.5 mg/day, 1 mg/day, 2.5 mg/day, 5 mg/day, 10 mg/day, 20 mg/day, 30 mg/day, 60 mg/day, 90 mg/day, 120 mg/day, 150 mg/day, 180 mg/day, 210 mg/day, 240 mg/day, 270 mg/day, 300 mg/day, 360 mg/day, 400 mg/day, 440 mg/day, 480 mg/day, 520 mg/day 580 mg/day, 600 mg/day, 620 mg/day, 640 mg/day, 680 mg/day, and 720 mg/day.

The administration dose of anti-CD20 antibodies, such as rituximab, should be tailored to the individual needs of the patient. For cancer treatment, rituximab is typically administered at a dose of 375 mg/m2 as an intravenous infusion. The initial dose is often followed by additional doses of 375 mg/m2, given once a week for a total of 4 to 8 weeks, depending on the specific treatment protocol and patient response. In maintenance therapy, rituximab may be administered at a dose of 375 mg/m2 every 2 to 3 months for up to 2 years. The infusion rate should start at 50 mg/h and can be increased by 50 mg/h increments every 30 minutes to a maximum of 400 mg/h, provided there are no severe infusion reactions. Premedication with acetaminophen and an antihistamine, such as diphenhydramine, is recommended to minimize the risk of infusion-related reactions.

The administration dose of rituximab should be tailored to the individual needs of the patient. For cancer treatment, rituximab is typically administered at a dose of 375 mg/m2 as an intravenous infusion. The initial dose is often followed by additional doses of 375 mg/m2, given once a week for a total of 4 to 8 weeks, depending on the specific treatment protocol and patient response. In maintenance therapy, rituximab may be administered at a dose of 375 mg/m2 every 2 to 3 months for up to 2 years. The infusion rate should start at 50 mg/h and can be increased by 50 mg/h increments every 30 minutes to a maximum of 400 mg/h, provided there are no severe infusion reactions. Premedication with acetaminophen and an antihistamine, such as diphenhydramine, is recommended to minimize the risk of infusion-related reactions.

In one example, the dose of rituximab for treating cancer, specifically B-cell nonHodgkin's lymphoma, can include (1) (Initial Dose) the initial dose of rituximab of 375 mg/m2, administered as an intravenous (IV) infusion, (2) (Subsequent Dose) this initial dose is often followed by additional doses of 375 mg/m2, given once a week for a total of 4 to 8 weeks, depending on the specific treatment protocol and the subject's response, and/or (3) (Maintenance Therapy) in some cases, rituximab may be administered as maintenance therapy. For example, in follicular lymphoma, rituximab may be given at a dose of 375 mg/m2 every 2 to 3 months for up to 2 years following the initial treatment regimen. When used in combination with other chemotherapy agents, the dosing schedule of rituximab may be adjusted based on the specific chemotherapy regimen. For instance, in combination with CHOP (cyclophosphamide, doxorubicin, vincristine, and prednisone) chemotherapy, rituximab is typically administered on day 1 of each chemotherapy cycle.

The term “treat” is used and includes both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

The pharmaceutical compositions can be included in a container, pack, or dispenser together with instructions for administration.

The composition of the present invention is capable of further forming salts. The composition of the present invention can form more than one salt per molecule, e.g., mono-, di-, tri-. All of these forms are also contemplated within the scope of the claimed invention.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane di sulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates), of the same salt.

As used herein, the term “selectively” means tending to occur at a higher frequency in one population than in another population. The compared populations can be cell populations. Preferably, a compound of the present invention, or a pharmaceutically acceptable salt or solvate thereof, acts selectively on a cancer or precancerous cell but not on a normal cell. Preferably, a compound of the present invention, or a pharmaceutically acceptable salt or solvate thereof, acts selectively to modulate one molecular target (e g., nucleotide excision repair (NER) players ERCC3). The invention also provides a method for selectively inhibiting the activity of an enzyme, such as NER proteins. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be the to occur selectively in cancer cells if it occurred greater than twice as frequently in cancer cells as compared to normal cells.

The composition, or pharmaceutically acceptable salts or solvates thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the invention can be found in Remington: the Science and Practice of Pharmacy, 19.sup.th edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

As used herein, a “subject in need thereof” is a subject having a precancerous condition. Preferably, a subject in need thereof has cancer. A “subject” includes a mammal. The mammal can be e.g., any mammal, e.g., a human, primate, bird, mouse, rat, dog, cat, cow, horse, goat, camel, sheep or a pig. Preferably, the mammal is a human. The subject of the present invention includes any human subject who has been diagnosed with, has symptoms of, or is at risk of developing a cancer or a precancerous condition.

A subject in need thereof may have refractory or resistant cancer. “Refractory or resistant cancer” means cancer that does not respond to treatment. The cancer may be resistant at the beginning of treatment or it may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In certain embodiments the prior therapy is monotherapy. In certain embodiments the prior therapy is combination therapy.

In some embodiments, a subject in need thereof may have a secondary cancer as a result of a previous therapy. “Secondary cancer” means cancer that arises due to or as a result from previous carcinogenic therapies, such as chemotherapy.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer results in a decrease in number and size of tumors. Preferably, after treatment, tumor number or size is reduced by 5% or greater relative to number prior to treatment; more preferably, tumor number or size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2x, 3x, 4x, 5x, 10x, or 50x.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2x, 3x, 4x, 5x, 10x, or 50x.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population receiving monotherapy with a drug that is not a compound of the present invention, or a pharmaceutically acceptable salt or solvate thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt or solvate thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. After treatment, tumor regrowth can be less than 5%; more preferably, tumor regrowth can be less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%;

One skilled in the art may refer to general reference texts for detailed descriptions of known techniques discussed herein or equivalent techniques. These texts can, of course, also be referred to in making or using an aspect of the invention.

### EXAMPLES

In order that the disclosure disclosed herein may be more efficiently understood, examples are provided below. It should be understood that these examples are for illustrative purposes only and are not to be construed as limiting the disclosure in any manner.

### Example 1

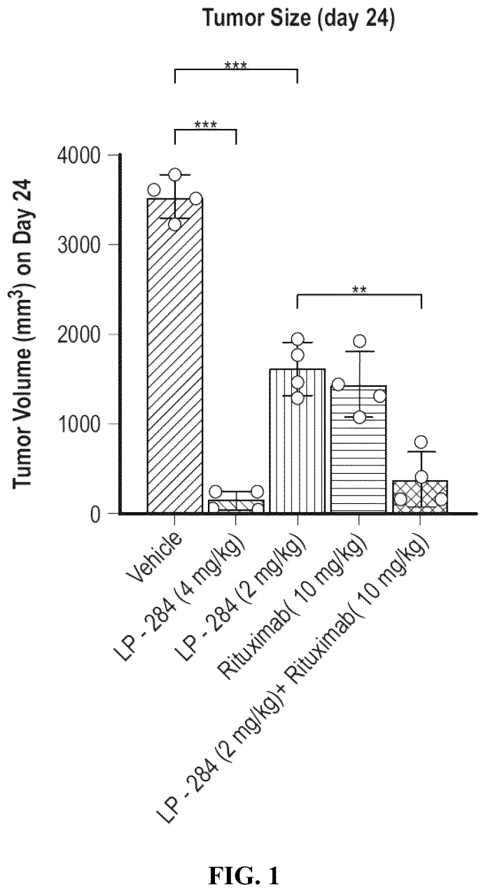

FIG. 1 shows the combined effect of LP-284 and an anti-CD20 monoclonal antibody (Rituximab) on tumor growth in a xenograft model. HRD genomic scars from 107 B-NHL patients in the Pan-Cancer Analysis of Whole Genomes (PCAWG) study were tested. At least 10% of B-NHL patients were likely HRD+, with approximately 90% of these cases being diffuse large B-cell lymphoma (DLBCL). LP-284 showed anti-tumor activity in preclinical models, inducing DNA DSB and cell apoptosis. In xenograft mice with OCLLY1 (TP53 mutated; DLBCL/HGBL-MYC/BCL2 double translocation), treatment with 4 mg/kg LP-284 resulted in 99% tumor growth inhibition (TGI) at day 24. Treatment with 2 mg/kg LP-284 alone resulted in 57% TGI, and in combination with Rituximab, resulted in 93% TGI at day 24. The calculated bliss synergy score between LP-284 and Rituximab on day 24 is 10, indicating synergy.

LP-284's efficacy in DLBCL/HGBL-MYC/BCL2 xenograft models was evaluated. LP-284, vehicle (saline), and Rituximab were administered intravenously in OCLLY1-derived tumor xenografts in mice. All vehicle-treated mice were sacrificed by day 24 due to excessive tumor size. LP-284 as a single agent or in combination with Rituximab significantly reduced tumor volume by day 24, as shown by tumor growth inhibition and final tumor volume measurements.

### Example 2

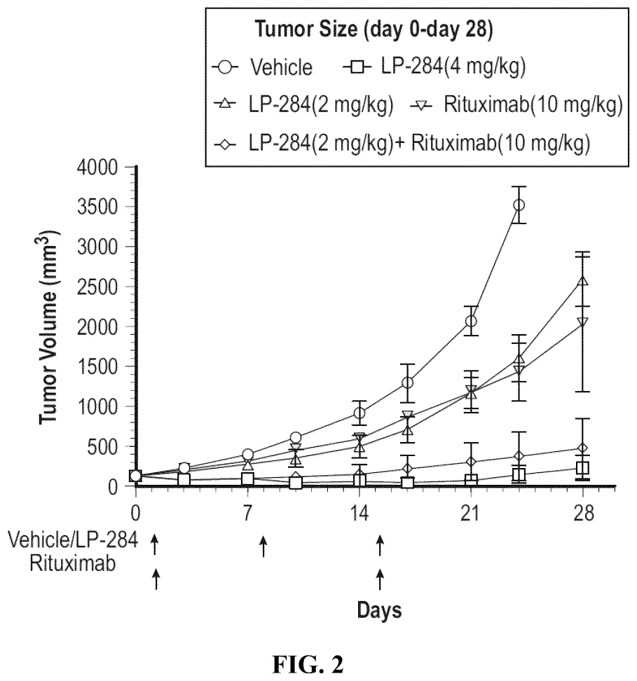

FIG. 2 shows the tumor growth curves during the entire study course from the same study as shown in FIG. 1. LP-284 was tested for anti-tumor efficacy as a monotherapy and in combination with rituximab in OCI-LY 1 DLBCL/HGBL-MYC/BCL2 cell line-derived tumor xenografts in mice. Methods: OCLLY1 cells were implanted subcutaneously in NOD.SCID mice. The following treatments were initiated when the tumors reached a volume of >100 mm3: vehicle (saline, administered intravenously on days 1, 8, and 15 in a 28-day cycle), 2 mg/kg or 4 mg/kg LP-284 (administered intravenously on days 1, 8, and 15 in a 28-day cycle), 10 mg/kg rituximab (administered intravenously on days 1 and 15 in a 28-day cycle), 2 mg/kg LP-284 and 10 mg/kg rituximab in combination. Four mice were used in each treatment group. Tumor Growth Inhibition (TGI) was calculated as % TGI=(1−((Tt/T0)/(Ct/C0)))/(1−(C0/Ct))*100 where Tt=mean tumor volume of treated at time t, TO=mean tumor volume of treated at time 0, Ct=mean tumor volume of control at time t, CO=mean tumor volume of control at time 0. The bliss synergy score was calculated as: SBliss=EA,B, . . . , N−100 (1−(1-EA100) (1−EB100) . . . (1−EN100)) where (1−EA,B, . . . , N100) represent probabilities that drugs A, B, . . . , N do not inhibit the target.

### Example 3

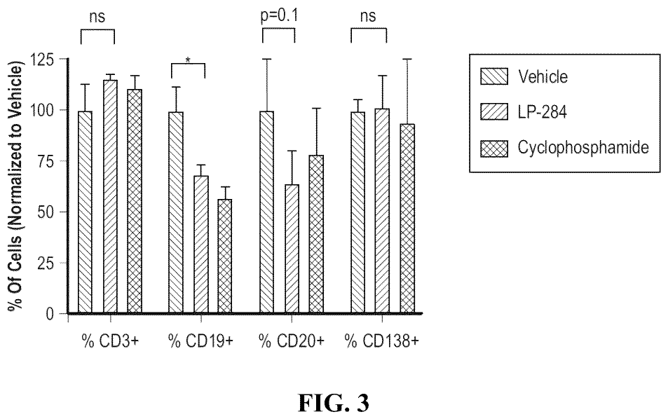

FIG. 3 shows that LP-284 treatment significantly reduced B-cells (CD 19+ or CD20+) by approximately 30% in immune-proficient mice after 24 days, comparable to the reduction achieved by cyclophosphamide. In this study, vehicle, LP-284 (4 mg/kg), and cyclophosphamide (25 mg/kg, a clinically relevant dose) were administered intravenously on days 1, 8, and 15, with three mice per treatment group. Blood samples were collected on Day 16 and analyzed by flow cytometry to quantify the B-cell populations. The results demonstrated that LP-284 was effective in reducing B-cells similarly to cyclophosphamide. Additionally, all mice maintained normal body weight, and no abnormal findings were observed during gross necropsy, indicating that LP-284 was well-tolerated.

What is claimed:

1. A method of treating a cancer characterized by malignant CD20-positive B cells in a subject in need thereof, the method comprising administering to the subject a therapeutically effective amount of:
(a) a hydroxyureamethyl acylfulvene or a pharmaceutically acceptable salt thereof, and
(b) an anti-CD20 antibody,
wherein the hydroxyureamethyl acylfulvene or a pharmaceutically acceptable salt thereof and the anti-CD20 antibody are administered in combination therapy.

2. The method of claim 1, wherein the cancer is a B-cell malignancy selected from the group consisting of B-cell non-Hodgkin's lymphoma and chronic lymphocytic leukemia.

3. The method of claim 2, wherein the B-cell non-Hodgkin's lymphoma is selected from the group consisting of diffuse large B-cell lymphoma (DLBCL), follicular lymphoma (FL), mantle cell lymphoma (MCL), Burkitt lymphoma, and double-hit lymphoma.

4. The method of claim 1, wherein the cancer cells are homologous recombination deficiency positive (HRD+).

5. The method of claim 4, further comprising determining that the subject is HRD+ by performing a genetic or genomic assay on a tumor sample from the subject prior to administering the hydroxyureamethyl acylfulvene and the anti-CD20 antibody.

6. The method of claim 1, wherein the anti-CD20 antibody is selected from the group consisting of rituximab, obinutuzumab, ofatumumab, and tositumomab.

7. The method of claim 1, wherein the hydroxyureamethyl acylfulvene is (+)-hydroxyureamethyl acylfulvene (LP-284).

8. The method of claim 1, wherein the hydroxyureamethyl acylfulvene is (−)-hydroxyureamethyl acylfulvene (LP-184).

9. The method of claim 1, wherein the anti-CD20 antibody is rituximab or an antigen-binding fragment or biosimilar thereof, the biosimilar being selected from the group consisting of blitzima, ritemvia, and tuxella.

10. The method of claim 1, wherein administration of the anti-CD20 antibody induces degradation of the nucleotide excision repair protein XPB/ERCC3 in the malignant B cells, thereby rendering the cells nucleotide excision repair deficient and sensitizing the cancer cells to the acylfulvene, and wherein the combined administration of the hydroxyureamethyl acylfulvene and the anti-CD20 antibody produces a synergistic inhibition of tumor growth relative to administration of either agent alone, as measured by a Bliss synergy score of at least about 10 in a xenograft model.

11. A pharmaceutical article of manufacture comprising:
(a) a therapeutically effective amount of a hydroxyureamethyl acylfulvene or a pharmaceutically acceptable salt thereof;
(b) a therapeutically effective amount of an anti-CD20 antibody or a pharmaceutically acceptable salt or antigen-binding fragment thereof; and
(c) at least one pharmaceutically acceptable carrier or excipient;
wherein the hydroxyureamethyl acylfulvene and the anti-CD20 antibody are provided in a form suitable for use in combination therapy for treating a B-cell cancer.

12. The pharmaceutical article of claim 11, wherein the hydroxyureamethyl acylfulvene is selected from the group consisting of (+)-hydroxyureamethyl acylfulvene (LP-284), (−)-hydroxyureamethyl acylfulvene (LP-184), and Irofulven.

13. The pharmaceutical article of claim 11, wherein the anti-CD20 antibody is selected from the group consisting of rituximab, obinutuzumab, ofatumumab, and tositumomab.

14. The pharmaceutical article of claim 11, wherein the article is a kit comprising:
(a) a first container comprising the hydroxyureamethyl acylfulvene or a pharmaceutically acceptable salt thereof;
(b) a second container comprising the anti-CD20 antibody or a pharmaceutically acceptable salt thereof; and
(c) printed instructions indicating that the contents of the first and second containers are to be administered to a subject with a B-cell non-Hodgkin's lymphoma as combination therapy.

15. The pharmaceutical article of claim 11, further comprising a second therapeutic agent selected from the group consisting of paclitaxel and cisplatinum.

16. A method of treating homologous recombination deficiency positive diffuse large B-cell lymphoma (DLBCL) in a subject in need thereof, the method comprising:
(a) intravenously administering to the subject (+)-hydroxyureamethyl acylfulvene (LP-284) at a dose of about 2 mg/kg to about 4 mg/kg on days 1, 8, and 15 of a 28-day treatment cycle; and
(b) intravenously administering to the subject rituximab at a dose of about 10 mg/kg on at least days 1 and 15 of the 28-day treatment cycle;
wherein the combined administration results in inhibition of tumor growth in the subject, and is characterized in that in an OCI-LY1 DLBCL xenograft model the same dosing regimen produces at least about 90% tumor growth inhibition by day 24 relative to vehicle-treated controls.

17. The method of claim 16, wherein the diffuse large B-cell lymphoma is a double-hit lymphoma comprising MYC and BCL2 translocations.

18. The method of claim 17, wherein the lymphoma cells further comprise a TP53 mutation.

19. The method of claim 16, wherein treatment further results in a reduction of at least about 30% in the number of circulating CD19+ and/or CD20+ B cells after three intravenous doses of LP-284, as measured by flow cytometry.

20. The method of claim 16, wherein the subject has relapsed or refractory B-cell non-Hodgkin's lymphoma following at least one prior therapy.
